(19) United States
(12) Patent Application Publication
(10) Pub. No.: US 2007/0196317 A1
(43) Pub. Date: Aug. 23, 2007

(54) Body Care Composition

(76) Inventor: Michael G. Ord, Austell, GA (US)

Correspondence Address: ADVANTIA LAW GROUP, 9035 SOUTH 1300 EAST, SUITE 200, SANDY, UT, 84094, US

(21) Appl. No.: 11/307,714

(22) Filed: Feb. 17, 2006

(51) Int. Cl. A61K 8/97; A61K 36/53; A61K 36/328

(52) U.S. Cl. 424/74; 424/745; 424/748; 424/769

(57) ABSTRACT

There is body care composition including botanical oils. The composition includes tea tree oil; lavender oil; myrrh; and/or sandalwood oil. There is a surfactant base such as but not limited to shampoo and/or a mild dishwashing detergent. The tea tree oil may be from about 2%, 5%, or 8% to about 15% by volume of the composition. The lavender oil may be from about 0.5%, 1%, 2%, or 4% to about 5% or 6% by volume of the composition. The myrrh may be from about 0.5% or 1% to about 5% by volume of the composition. The sandalwood oil may be from about 0.5% or 1% to about 5% by volume of the composition.

### BACKGROUND OF THE INVENTION

1. Field of the Invention

The present invention relates to body care compositions, specifically body care compositions including botanical oils.

2. Description of the Related Art

Hair is an appendage of the scalp and skin and serves no vital function. However, the psychological impact on an individual can be immense. Loss of hair can be very detrimental to an individual's self esteem.

Hair grows in what is known as the Pilar cycle. There are three phases to the Pilar cycle. The first phase, the anagen phase, is the growth or activity stage of the cycle. The catagen phase is a regressive stage. The telogen phase is a resting phase. Further information may be found in U.S. Pat. No. 6,747,008 which is incorporated by reference herein in its entirety.

It is generally agreed that Sebum and Dihydrotestosterone are culprits in disrupting the Pilar cycle, thus leading to hair loss. More information may be found in U.S. Pat. No. 5,750,108, which is incorporated by reference herein in its entirety. Such may interfere with normal hair production by binding to important components of the Pilar cycle, blocking follicles, and/or damaging follicle cells.

Other body problems include but are not limited to nail fungus and dandruff of the scalp.

Methods and compositions have been developed to address one or more of these problems. In particular, U.S. Pat. No. 6,043,202 discloses a shampoo composition, an oil composition and a method for the treatment of cradle cap (seborrhea dermatitis) primarily in infants and small children. In the method, the scalp of an infant is treated with the oil followed by gentle brushing and the scalp is then contacted with the shampoo and water. Usually the cradle cap condition is eliminated in 1 to 5 days. A kit including the oil, shampoo, sponge, brush, fine toothed comb and optional separate instructions are described.

More, in U.S. Pat. No. 4,832,947, there is disclosed a permanent waving process in which an essential oil is incorporated into hair fibers. The incorporated of the essential oil into the hair fiber not only provides capacity to remove bad odors resulting from freshly permanent waved hair but also gives to the hair a supple and smooth feeling. The essential oil is incorporated in the oxidizing or neutralizing solution of the permanent waving process. The essential oils employed are those containing at least one component, ingredient or constituent having an ethylenically unsaturated bond, i.e. ##STR1 ##.

Others include that disclosed in U.S. Pat. Nos. 5,350,774 and 4,999,187. In some compositions, application is inconvenient, difficult to use, expensive, non-portable, harsh, and/or ineffective. In some instances a composition may fail to encourage stronger and/or thicker hair, fail to lessen hair loss, fail to contribute to stronger hair retention, fail to lessen nail fungus, fail to lessen scalp dandruff, fail to promote thick, healthy, shiny, silky, attractive, beautiful, manageable, and/or soft hair.

What is needed is a body care product that solves one or more of the problems described herein and/or one or more problems that may come to the attention of one skilled in the art upon becoming familiar with this specification.

### SUMMARY OF THE INVENTION

The present invention has been developed in response to the present state of the art, and in particular, in response to the problems and needs in the art that have not yet been fully solved by currently available body care compositions. Accordingly, the present invention has been developed to provide a body care composition including botanical oils.

In one embodiment, there is body care composition including botanical oils. The composition may include tea tree oil; lavender oil; myrrh; and/or sandalwood oil. There may be a surfactant base such as but not limited to shampoo and/or a mild dishwashing detergent. The tea tree oil may be from about 2%, 5%, or 8% to about 15% by volume of the composition. The lavender oil may be from about 0.5%, 1%, 2%, or 4% to about 5% or 6% by volume of the composition. The myrrh may be from about 0.5% or 1% to about 5% by volume of the composition. The sandalwood oil may be from about 0.5% or 1% to about 5% by volume of the composition.

Reference throughout this specification to features, advantages, or similar language does not imply that all of the features and advantages that may be realized with the present invention should be or are in any single embodiment of the invention. Rather, language referring to the features and advantages is understood to mean that a specific feature, advantage, or characteristic described in connection with an embodiment is included in at least one embodiment of the present invention. Thus, discussion of the features and advantages, and similar language, throughout this specification may, but do not necessarily, refer to the same embodiment.

Furthermore, the described features, advantages, and characteristics of the invention may be combined in any suitable manner in one or more embodiments. One skilled in the relevant art will recognize that the invention can be practiced without one or more of the specific features or advantages of a particular embodiment. In other instances, additional features and advantages may be recognized in certain embodiments that may not be present in all embodiments of the invention.

These features and advantages of the present invention will become more fully apparent from the following description and appended claims, or may be learned by the practice of the invention as set forth hereinafter.

### DETAILED DESCRIPTION OF THE INVENTION

For the purposes of promoting an understanding of the principles of the invention, reference will now be made to the exemplary embodiments illustrated in the drawings, and specific language will be used to describe the same. It will nevertheless be understood that no limitation of the scope of the invention is thereby intended. Any alterations and further modifications of the inventive features illustrated herein, and any additional applications of the principles of the invention as illustrated herein, which would occur to one skilled in the relevant art and having possession of this disclosure, are to be considered within the scope of the invention.

Reference throughout this specification to “one embodiment,” “an embodiment,” or similar language means that a particular feature, structure, or characteristic described in connection with the embodiment is included in at least one embodiment of the present invention. Thus, appearances of the phrases “one embodiment,” “an embodiment,” and similar language throughout this specification may, but do not necessarily, all refer to the same embodiment, different embodiments, or component parts of the same or different illustrated invention. Additionally, reference to the wording “an embodiment,” or the like, for two or more features, elements, etc. does not mean that the features are related, dissimilar, the same, etc. The use of the term “an embodiment,” or similar wording, is merely a convenient phrase to indicate optional features, which may or may not be part of the invention as claimed.

Each statement of an embodiment is to be considered independent of any other statement of an embodiment despite any use of similar or identical language characterizing each embodiment. Therefore, where one embodiment is identified as “another embodiment,” the identified embodiment is independent of any other embodiments characterized by the language “another embodiment.” The independent embodiments are considered to be able to be combined in whole or in part one with another as the claims and/or art may direct, either directly or indirectly, implicitly or explicitly.

Finally, the fact that the wording “an embodiment,” or the like, does not appear at the beginning of every sentence in the specification, such as is the practice of some practitioners, is merely a convenience for the reader's clarity. However, it is the intention of this application to incorporate by reference the phrasing “an embodiment,” and the like, at the beginning of every sentence herein where logically possible and appropriate.

In order to demonstrate the practice of the present invention, the following examples have been prepared. The examples should not, however, be viewed as limiting the scope of the invention. The claims will serve to define the invention

In one embodiment, there is body care composition including botanical oils. The composition may include a surfactant; tea tree oil (in one example known as Melaleuca Alternafolia); lavender oil (in one example known as Lavandula Angustifolia); myrrh (in one example known as Commiphora Myrrha); and/or sandalwood oil (in one example known as Santalum album). In one example, there is a composition including tea tree oil, lavender oil, and myrrh. In another example, there is a composition including a shampoo base, tea tree oil, lavender oil, myrrh, and sandalwood oil.

The surfactant or surfactant base may be shampoo and/or a mild dishwashing detergent. It is understood that the numbers and types of surfactants are plethoric. A surfactant may also be an emulsifier and or may be basic. In one non-limiting example a surfactant includes water, sodium laureth sulfate, cocamidopropyl betaine, sodium olefin C14-16 Sulfonate, cocamide mea, dimethicone copolyol, trimethylsilyamodimethicone, laureth-4, laureth-23, cyclopentaxiloxane, citric acid, tetrasodium edta, and methylchloroisothiazolinone. Fragrances and/or coloring agents may be added.

The tea tree oil may be from about 2%, 5%, or 8% to about 15% by volume of the composition. It is understood that above about 15% by volume, tea tree oil may be an irritant to some individuals. Accordingly, while it is anticipated that amounts greater than about 15% may be used in the present invention, it is preferred that the amount be less than about 15% by volume.

The lavender oil may be from about 0.5%, 1%, 2%, or 4% to about 5% or 6% by volume of the composition. It is preferred that lavender oil be present in an amount from about 4% to about 6% by volume. It is theorized that lavender oil plays a role in penetration and in widening pores, in particular in dermal penetration. However, lavender oil may play other roles, and therefore the penetrating theory of lavender oil is not meant to be limiting.

The myrrh may be from about 0.5% or 1% to about 5% by volume of the composition. It is theorized that myrrh play an antibiotic role. However, myrrh may play other roles, and therefore the antibiotic role of myrrh is not meant to be limiting.

The sandalwood oil may be from about 0.5% or 1% to about 5% by volume of the composition. Sandalwood oil is theorized to play a role in making hair supple. However, sandalwood oil may play other roles, and therefore the supple theory of sandalwood is not meant to be limiting.

In one example there is a single step treatment for the hair, scalp, skin, and/or nails which is used as a shampoo, soap, application, ointment, salve, balm, etc. The treatment may include using the composition to treat and/or clean an area of the body and may be directed to stimulate hair growth, fight nail fungus, and/or fight dandruff. A composition may be applied to an area such as but not limited to the hair, scalp, and forehead, and may be left thereon for about 5 minutes. A composition may be lathered. A composition may be lathered after an initial application time. A composition may be used on an entire body. A composition may be applied to finger and/or toenails directly and may be left thereon for a period of time such as but not limited to about 5 minutes.

It is preferred that a composition applied to the hair and/or scalp include a surfactant. It is preferred that a composition applied to a fingernail or a toenail include at least tea tree oil, lavender oil, and myrrh.

It is understood that the above-described preferred embodiments are only illustrative of the application of the principles of the present invention. The present invention may be embodied in other specific forms without departing from its spirit or essential characteristics. The described embodiment is to be considered in all respects only as illustrative and not restrictive. The scope of the invention is, therefore, indicated by the appended claim rather than by the foregoing description. All changes which come within the meaning and range of equivalency of the claims are to be embraced within their scope.

Thus, while the present invention has been fully described above with particularity and detail in connection with what is presently deemed to be the most practical and preferred embodiment of the invention, it will be apparent to those of ordinary skill in the art that numerous modifications, including, but not limited to, variations in size, materials, shape, form, function and manner of operation, assembly and use may be made, without departing from the principles and concepts of the invention as set forth in the claims.

What is claimed is:

1. A body care composition including botanical oils, comprising:
tea tree oil;
lavender oil; and
myrrh.

2. The body care composition of claim 1, further comprising sandalwood oil.

3. The body care composition of claim 1, further comprising a surfactant base.

4. The body care composition of claim 3, wherein the surfactant base comprises a shampoo.

5. The body care composition of claim 3, wherein the surfactant base comprises a mild dishwashing detergent.

6. The body care composition of claim 2, wherein:
the tea tree oil comprises 6%-15% by volume of the composition;
the lavender oil comprises 4%-6% by volume of the composition;
the myrrh comprises 1%-5% by volume of the composition; and
the sandalwood oil comprises 1%-5% by volume of the composition.

7. A body care composition including botanical oils, consisting essentially of:
tea tree oil;
lavender oil;
myrrh; and
sandalwood oil.

8. The body care composition of claim 6, wherein:
the tea tree oil comprises 6%-15% by volume of the composition;
the lavender oil comprises 4%-6% by volume of the composition;
the myrrh comprises 1%-5% by volume of the composition; and
the sandalwood oil comprises 1%-5% by volume of the composition.

9. A body care composition including botanical oils, consisting essentially of:
a surfactant base;
tea tree oil;
lavender oil;
myrrh; and
sandalwood oil.

10. The body care composition of claim 8, wherein the surfactant base comprises a shampoo.

11. The body care composition of claim 8, wherein the surfactant base comprises a mild dishwashing detergent.
